(19) United States
(12) Patent Application Publication
(10) Pub. No.: US 2025/0049915 A2
(43) Pub. Date: Feb. 13, 2025

(54) NANOPARTICULATE FORMULATION

(71) Applicant: NewImmune II, LLC, Wilmington, DE (US)

(72) Inventors: Brian HORSBURGH, New Canaan, CT (US); Mark David MOODY, Concord, MA (US)

(73) Assignee: NewImmune II, LLC, Wilmington, DE (US)

(21) Appl. No.: 18/700,024

(22) PCT Filed: Oct. 11, 2022
(86) PCT No.: PCT/GB2022/052569

§ 371 Date: Apr. 10, 2024

Related U.S. Application Data
(60) Provisional application No. 63/321,886, filed on Mar. 21, 2022.
(60) Provisional application No. 63/254,253, filed on Oct. 11, 2021.

(30) Foreign Application Priority Data
Feb. 2, 2022 (GB) 2201351.0

(51) Int. Cl. A61K 39/39; A61K 39/00; A61K 39/215; A61K 47/69

(52) U.S. Cl. CPC A61K 39/39; A61K 9/1271; A61K 31/4745; A61K 38/2013; A61K 39/215; A61K 47/10; A61K 47/24; A61K 47/28; A61K 47/6951; A61P 35/00; A61K 2039/55511; A61K 2039/55555

(57) ABSTRACT

The present disclosure relates to nanoparticulate vaccine adjuvants, and to vaccine compositions which contain nanoparticulate vaccine adjuvants; to methods of preparing such adjuvants and compositions; and to methods of using such compositions and adjuvants for vaccination. The vaccine adjuvants disclosed herein are effective for enhancing the immune response to vaccination.

### FIELD OF THE DISCLOSURE

The present disclosure relates to nanoparticulate vaccine adjuvants, and to vaccine compositions which contain nanoparticulate vaccine adjuvants; to methods of preparing such adjuvants and compositions; and to methods of using such compositions and adjuvants for vaccination. The vaccine adjuvants disclosed herein are effective for enhancing the immune response to vaccination.

### BACKGROUND

Vaccination is an important public health measure, as underlined by the recent SARS-COV-2 pandemic. An effective vaccine against an infectious disease, if widely administered across a population, can both slow the transmission of the disease and reduce the severity of the symptoms experienced by the vaccinated population. Vaccines can also be effective for treating certain types of disease, including proliferative disorders such as cancer.

Vaccines work by inducing an immune response which operates to protect the vaccinated individual. The immune system is stimulated by exposure to a suitable immunogen, such as a pathogen antigen or a cancer-associated antigen, which, directly or indirectly, excites a protective adaptive immune response. The adaptive immune response may be mediated by B cells and/or T cells. The objective may be to provide long-term immunity against the antigen and/or pathogens, cells or entities bearing that antigen.

Several different types of vaccines have been developed. Vaccines developed for protection against infectious disease include inactivated and live-attenuated vaccines; toxoid vaccines; viral vector vaccines; subunit, recombinant, polysaccharide, and conjugate vaccines; and mRNA vaccines. Vaccines developed for protection against cancer include autologous patient-derived immune cell vaccines, tumor antigen-expressing recombinant virus vaccines, peptide vaccines, DNA vaccines, and heterologous whole-cell vaccines derived from established human tumor cell lines. In each case, the vaccine contains either antigens capable of inducing an immune response, or polynucleotides encoding antigens that are capable of inducing an immune response.

Many vaccines also contain adjuvant ingredients. These are substances that are capable of enhancing the immune response induced by the vaccine. This can be important, particularly where the vaccine antigen has low immunogenicity, or where the vaccine is to be administered to immunologically compromised, immunologically depleted or immunologically immature patients, such as infants or the elderly. The bolstering effect of an adjuvant can also allow the dose of vaccine per patient to be reduced, which is important for vaccine sparing in situations where there is insufficient vaccine. Vaccine adjuvants approved for human use include aluminium-based mineral salts (Alum), MF59, monophosphoryl lipid A (MPL) and a CpG oligodeoxynucleotide (CpG 1018).

Another recognised vaccine adjuvant is imiquimod (R-837). Imiquimod (R-837)—also known as 1-(2-methylpropyl)imidazo[4,5-c]quinolin-4-amine (CAS Number: 99011-02-6), R-837, and S-26308—is a small molecule imidazoquinoline drug having the structural formula:

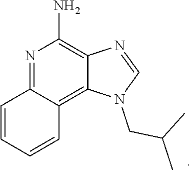 [SMILES: CC(C)Cn1cnc2c(N)nc3ccccc3c21]

Various active structural analogues of imiquimod (R-837) have been synthesized and characterised. These include the imidazoquinolines resiquimod (R-848), gardiquimod, CL097, S28690, 852-A, and 854A; the thiazoloquinolone CL075; and others, including those shown in Table 1 below:

TABLE 1

| Structural analogue | Molecular structure |
|---|---|
| Resiquimod | 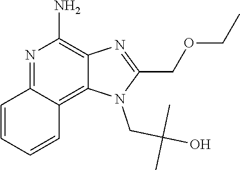 [SMILES: CCOCc1nc2c(N)nc3ccccc3c2n1CC(C)(C)O] |
| Gardiquimod | 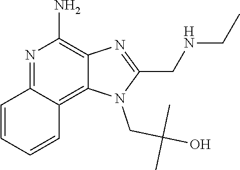 [SMILES: CCNCc1nc2c(N)nc3ccccc3c2n1CC(C)(C)O] |
| CL097 | 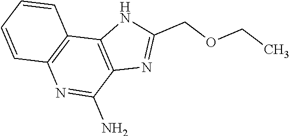 [SMILES: CCOCc1nc2c(N)nc3ccccc3c2[nH]1] |
| 852-A (PF-4878691) | 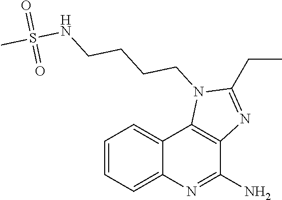 [SMILES: CCc1nc2c(N)nc3ccccc3c2n1CCCCNS(C)(=O)=O] |
| 854A (3M-011) | 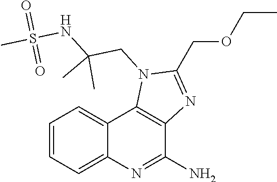 [SMILES: CCOCc1nc2c(N)nc3ccccc3c2n1CC(C)(C)NS(C)(=O)=O] |
| CL075 (3M-002) | 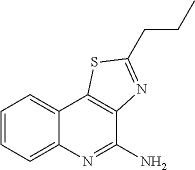 [SMILES: CCCc1nc2c(N)nc3ccccc3c2s1] |

Another exemplary structural analogue of imiquimod (R-837) is S28690, a small molecule TLR7 agonist, described in Hicks et al, Blood (2004) 104 (11): 3481.

These structural analogues of imiquimod (R-837) are active TLR7/8 ligands which have similar properties and activity to imiquimod (R-837), optionally including pH-dependent solubility. In this disclosure, the term “imiquimod” henceforth denotes imiquimod (R-837); but also embraces and refers to structural analogues of imiquimod (R-837) which are TLR7/8 agonists or TLR7/8 ligands, including but not limited to those listed above. Suitably, structural analogues of imiquimod (R-837) may display pH-dependent solubility, with increased solubility at lower pH. The term “imiquimod” as used herein accordingly includes imidazoquinolines which are active TLR7/8 ligands and which have the basic molecular structure:

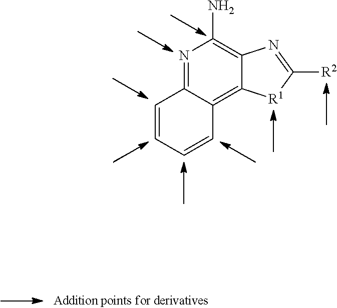

where R¹ is typically N and R² is typically H or C and where the imidazoquinoline is optionally substituted at one or more of the indicated addition points with one or more substituents, which substituents may be independently selected from branched, linear or cyclic alkyl, alkenyl, alcohol, alkylamine, alkoxy or alkoxyalkyl groups, in particular, C₁₋₁₀ alkyl, alkenyl, alcohol, alkylamine, alkoxy or alkoxyalkyl groups, or hydroxyl groups, or amine groups, or N-(C₁₋₁₀ alkyl)methanesulphonamide groups. The term “imiquimod” as used herein also includes close structural derivatives of these imidazoquinolines, including thiazoloquionoline derivatives, which are active TLR7/8 ligands.

Imiquimod stimulates the innate and adaptive immune system by activating toll-like receptors 7 and/or 8 (TLR7/8). It is approved by the FDA as an active ingredient in two topical cream formulations, Aldara® and Zyclara®.

Various studies have shown that topical imiquimod is capable of enhancing the immune response induced by a vaccine. In particular, topical imiquimod enhances both antibody responses and cellular responses to subcutaneous immunisation using ovalbumin; with the immune response shifted towards a Th1 phenotype with marked enhancement of IgG2a, IgG2b and CD8+ T cell responses (Johnston et al, Vaccine 2006 Mar. 10; 24 (11): 1958-65). Pre-treatment with topical imiquimod also significantly improves the immunogenicity of influenza vaccination in both young and elderly individuals (Hung et al, Lancet Infect Dis. 2016 February; 16 (2): 209-18). Similar results were reported by Adams et al, J. Clin. Oncol. 25 (18) suppl. 8545, which evaluated the safety and adjuvant activity of imiquimod when administered with a NY-ESO-1 protein vaccine.

These studies demonstrate that imiquimod is effective as a vaccine adjuvant. Topical application of imiquimod in the form of a cream, as reported in these studies, is however inconvenient and is not feasible for routine vaccination use. Topical application raises compliance issues, since the cream must remain on the skin for several hours in order to take effect. It may give rise to skin irritation, or be poorly tolerated by patients for other reasons. Consistency of imiquimod delivery is also a problem. For practical purposes, it would be preferable to formulate the adjuvant together with other vaccine ingredients for administration as a single vaccine formulation. It would be preferable to formulate the adjuvant as an injectable composition.

Formulating imiquimod for injection is not, however, a straightforward endeavour. Imiquimod has very low solubility in aqueous solution at physiological pH. FIG. 1 illustrates the aqueous solubility of imiquimod as a function of pH using Britton-Robinson buffer; showing that imiquimod has maximum solubility in aqueous solution at pH 2 or below (Chollett et al Pharm. Dev. Technol. 1999 January; 4 (1): 35-43). The aqueous solubility of imiquimod decreases sharply with increasing pH above pH 2, and it is only sparingly soluble above pH 6. For this reason, efforts to develop aqueous or aqueous-based formulations of imiquimod have typically involved the use of acidic solvents below pH 4, which is not acceptable for injection. Hayashi et al (Int. J. Urol. 2010 May; 17 (5): 483-90) describes a formulation of imiquimod which uses 0.1 M lactic acid, poloxamer, and HP-β-CD at low acidic pH. Guedes et al, (J. Braz. Chem. Soc., Vol. 31, No. 8, 1732-1745, 2020) similarly describes the solubilisation of imiquimod in β-cyclodextrin in the presence of citric acid at pH 3. Ramineni et al (J. Pharm. Sci. 2013 February; 102 (2): 593-603) developed mucoadhesive films containing imiquimod and HP-β-CD, using a mixture of acetate buffer and methanol at pH 4. Fox et al (Journal of Nanobiotechnology 2014, 12: 17) meanwhile discloses an anionic liposomal formulation comprising imiquimod solubilised in a lactic acid core at pH 2.5-3.5.

Whilst imiquimod can be formulated in aqueous-based formulations at low pH as described in these and similar references, this approach does not readily allow for co-formulation of the imiquimod with vaccine antigens and polynucleotides which denature or change their form at low pH. Low pH formulations are also unsuitable for parenteral administration.

In an alternative approach, polylactide (PLA)-based micelles have been core-loaded with imiquimod and surface-functionalized with an antigenic protein (HIV-1 Gag p24) for antigen delivery purpose (Jiménez-Sánchez, et al Pharm. Res. (2015) 32: 311-320). The imiquimod is encapsulated in the hydrophobic PLA core, whilst the p24 antigen is covalently attached through lysine and N-terminal amine groups to the N-succinimidyl pendant groups of the micelle corona, The release of imiquimod from the particles was however found to be very rapid (50% in 1 h, ˜75% in 4 to 5 hours). A nanomedicine format that provides a slower release of the adjuvant is thought to be likely to support a more potent immune response.

Against this backdrop, the present inventors have successfully developed and describe herein a nanoparticulate vaccine adjuvant that comprises imiquimod, is suitable for injection, and is capable of providing sustained release of imiquimod over an extended period of time, which can improve the performance of imiquimod as a vaccine adjuvant. The disclosed nanoparticulate vaccine adjuvant can be formulated with existing vaccines or co-formulated with vaccine antigens and/or polynucleotides and, if required, with additional vaccine ingredients; thereby also providing a vaccine composition comprising an imiquimod vaccine adjuvant which can be parenterally administered, including by injection.

### SUMMARY OF THE DISCLOSURE

According to one aspect, the present disclosure provides a vaccine adjuvant comprising a plurality of nanoparticles that comprise an outer lipid shell and an inner aqueous core encapsulated within the outer lipid shell; wherein the inner aqueous core comprises imiquimod and a host molecule that is capable of reversibly forming a complex with imiquimod. The vaccine adjuvant may be or may comprise an aqueous solution, aqueous dispersion or aqueous suspension of nanoparticles as disclosed, or may be or may comprise a dried or lyophilised preparation that can be hydrated to produce an aqueous solution, aqueous dispersion or aqueous suspension of nanoparticles as disclosed. The inner aqueous core may have a pH of about 6.5 or above, and/or may comprise a hydrogel. The imiquimod and host molecule may be dispersed within the hydrogel.

In a further aspect, the present disclosure provides a vaccine composition comprising (a) an antigen that is capable of inducing an immune response and/or a polynucleotide that encodes an antigen capable of inducing an immune response; and (b) a vaccine adjuvant comprising nanoparticles in accordance with the present disclosure. The vaccine composition may be or may comprise an aqueous solution, aqueous dispersion or aqueous suspension of components (a) and (b) as disclosed, or may be or may comprise a dried or lyophilised preparation that can be hydrated to produce an aqueous solution, dispersion or suspension of components (a) and (b) as disclosed. In some embodiments, some or all of the antigen and/or the polynucleotide of component (a) may be releasably attached to, associated with and/or encapsulated within the outer lipid shell of the nanoparticles of component (b). Component (a) may, additionally or alternatively, comprise a delivery vehicle, such as a nanoparticulate delivery vehicle, such as a plurality of nanoparticles that comprise an outer lipid shell and an inner aqueous core encapsulated within the outer lipid shell, where the antigen and/or polynucleotide is loaded into or onto the delivery vehicle.

The vaccine composition may comprise additional ingredients and excipients, including further adjuvants. The vaccine composition may, in some embodiments, comprise some or all of the active and/or excipient ingredients of a vaccine formulation which has been developed for prophylactic or therapeutic use; such as an approved vaccine formulation. Conveniently, the approved vaccine formulation may comprise an antigen capable of inducing an immune response and/or a polynucleotide that encodes an antigen capable of inducing an immune response. The vaccine composition of the present disclosure may comprise an approved vaccine formulation that includes an antigen capable of inducing an immune response and/or a polynucleotide that encodes an antigen capable of inducing an immune response, supplemented with a vaccine adjuvant comprising nanoparticles in accordance with the present disclosure.

In a further aspect, the present disclosure provides vaccine adjuvants as disclosed for use in a method of enhancing an immune response to a vaccine in a subject, such as a human subject. The present disclosure further provides a method of enhancing an immune response to a vaccine in a subject, such as a human subject, comprising the step of administering to the subject a vaccine adjuvant as herein disclosed, where the vaccine adjuvant is administered to the subject before, simultaneously with, and/or after administration of the vaccine. The vaccine may be any vaccine that is capable of inducing an immune response in a subject. The immune response may, for example, be a protective immune response, which is capable of protecting the subject against a disease, disorder or pathogen, including an infectious or proliferative disease or disorder, or a viral, bacterial or fungal pathogen. The immune response may be a therapeutic immune response, which is capable of alleviating or reducing the symptoms or manifestation of a disease or disorder, including an infectious disease or disorder.

In a further aspect, the present disclosure provides vaccine compositions as disclosed for use in a method of inducing an immune response in a subject, such as a human subject. The present disclosure further provides a method of inducing an immune response in a subject, such as a human subject, comprising the step of administering to the subject a vaccine composition as herein disclosed. The immune response may, for example, be a protective immune response, which is capable of protecting the subject against a disease, disorder or pathogen, including an infectious or proliferative disease or disorder, or a viral, bacterial or fungal pathogen. The immune response may be a therapeutic immune response, which is capable of alleviating or reducing the symptoms or manifestation of a disease or disorder, including an infectious disease or disorder.

The present disclosure further provides a method for manufacturing a vaccine adjuvant as disclosed, comprising the sequential steps of:
(a) solubilising imiquimod with the host molecule in aqueous solution at a pH buffered to about pH 6 or below; preferably to about pH 4-6, or to about pH 4.5-6, or to about pH 4-5.5, or to about pH 5-6;
(b) combining the resulting aqueous solution with lipids to form lipid shell nanoparticles encapsulating imiquimod; and then
(c) increasing the buffered pH of the formulation to about pH 6.5 or above, or to pH 7 or above, or to about pH 6.5-9 or to about pH 6.5-8.5, or to about pH 6.5-8; or to about pH 7-9 or to about pH 7-8.5 or to about pH 7-8 or to about pH 7.5-9.

The disclosure provides a method for manufacturing a vaccine composition as disclosed, comprising the step of combining a vaccine adjuvant as disclosed with an antigen that is capable of inducing an immune response and/or with a polynucleotide that encodes an antigen capable of inducing an immune response. This method may comprise combining a vaccine adjuvant as disclosed with an approved vaccine formulation that includes an antigen that is capable of inducing an immune response and/or a polynucleotide that encodes an antigen capable of inducing an immune response. The disclosure provides a method for manufacturing a vaccine composition as disclosed, comprising the steps of manufacturing a vaccine adjuvant according to the methods disclosed herein and adding an antigen that is capable of inducing an immune response and/or a polynucleotide that encodes an antigen capable of inducing an immune response, during or after step (a).

The disclosed vaccine adjuvants and vaccine compositions, buffered to physiological pH, may be administered to a subject parenterally, optionally by injection; and may be used for or in the course of immunisation against a wide range of diseases, disorders and pathogens, including infectious diseases and pathogens, and cancer.

The compositions of the present disclosure have been designed to facilitate controlled, sustained and optionally localised delivery and/or release of imiquimod, for the purpose of enhancing an immune response to a vaccine. The disclosed adjuvants are capable of providing sustained delivery and/or release of imiquimod over a period of time which allows for improved enhancement of the immune response.

The disclosed vaccine adjuvant comprises the small molecule imiquimod complexed with a host molecule and encapsulated in a liposome. Imiquimod (R-837) is contained in approved and marketed drugs (Aldara® and Zyclara®) and is well characterised and well understood by medical oncologists. Studies and clinical trials have shown the efficacy of imiquimod as a vaccine adjuvant, although it has not previously been formulated as herein disclosed for convenient parenteral administration and for controlled delivery and release.

### BRIEF DESCRIPTION OF THE FIGURES

FIG. 1 shows a graph illustrating the effect of pH on the solubility of imiquimod in aqueous solution.

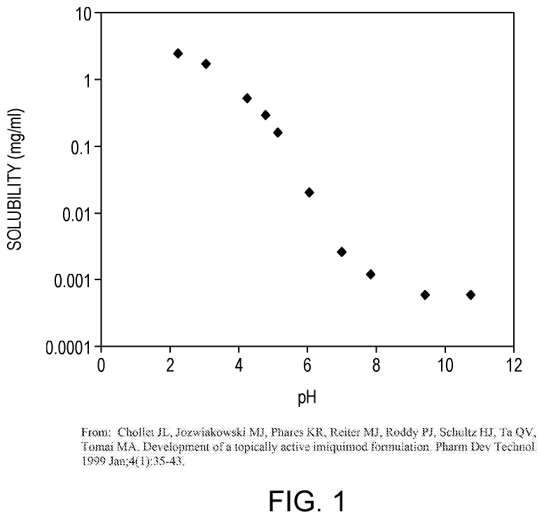

FIG. 2 shows the structure of an exemplary vaccine adjuvant nanoparticle according to the present disclosure.

FIG. 3 shows a Cryo TEM image of vaccine adjuvant nanoparticles according to the present disclosure.

FIG. 4 shows a flow chart illustrating the steps of an exemplary method for production of a vaccine adjuvant according to the present disclosure.

FIG. 5 shows the results of a release assay illustrating the release profile of imiquimod from a vaccine adjuvant according to the present disclosure.

### DEFINITIONS

Terms used in the present disclosure should, unless otherwise indicated, be understood to have their normal meanings in the art. In particular, “antigen” shall include any molecule or entity that is capable of inducing an immune response in vivo, in humans and/or animals. “Polynucleotide” shall include any nucleic acid containing more than one nucleotide. “Immune response” shall include any protective or defensive reaction of the immune system to an antigen, including innate, adaptive and responsive immune responses, Type 1 and Type 2 responses, cell-mediated and humoral and inflammatory immune responses. Within the context of the present disclosure, an “immune response” may usually be understood to mean a protective or therapeutic immune response, and/or an immune response that is effective to protect against or treat a disease, disorder or medical condition. “Immunisation” shall include the process of inducing an immune response, particularly a protective immune response, to an antigen in a subject; and a subject is “immune” after effective immunisation. A “vaccine” is a substance or composition that can be used to immunise a subject. A “vaccine adjuvant” is a substance that can be effective to expand, enhance, amplify, modulate, increase or in any way improve the immune response induced by a vaccine. A “hydrogel” is a matrix of water-swellable hydrophilic polymers, optionally cross-linked; “hydrogel polymer” is construed accordingly. The term “imiquimod” denotes imiquimod (R-837); but also embraces and refers to structural analogues of imiquimod (R-837) as defined hereinabove, which are TLR7/8 agonists or TLR7/8 ligands and which may display pH-dependent solubility, including but not limited to imidazoquinolines and thiazoloquinolones as shown in Table 1.

### DETAILED DESCRIPTION

The present disclosure provides a vaccine adjuvant comprising a plurality of nanoparticles, each nanoparticle comprising an outer lipid shell and an inner aqueous core encapsulated within the outer lipid shell; wherein the inner aqueous core comprises imiquimod and a host molecule that is capable of reversibly forming a complex with imiquimod. The aqueous core may comprise imiquimod complexed with a host molecule. The aqueous core of the nanoparticle may comprise uncomplexed imiquimod and/or host molecule.

The inner aqueous core may, optionally, comprise a hydrogel. The imiquimod and host molecule may, optionally, be dispersed, dissolved or suspended in the hydrogel.

The inner aqueous core may have a pH of about 6.5 or above. Suitably, the inner aqueous core may have a pH of at least 7, or a pH of at least 7.5. The inner aqueous core may have a pH of no more than about pH 9 or no more than about pH 8.5; or may suitably have a pH between about pH 6.5-9 or about 6.5-8.5 or about 6.5-8 or between about pH 7-9 or between about pH 7-8.5 or between about pH 7-8 or between about pH 7.5-9.

The outer lipid shell of the nanoparticle comprises one or more lipid layers or bilayers, which enclose a central core. The lipids forming the shell may be neutral, zwitterionic, anionic or cationic lipids at physiologic pH. The lipids within and/or between each lipid layer or bilayer, may, optionally, be cross-linked. The outer lipid shell may accordingly be composed of one or more concentric lipid layers, optionally crosslinked, wherein the lipids can be neutral, anionic or cationic lipids at physiologic pH. The composition of the lipid shell and the extent of cross-linking within or between the lipid layers can be varied in order to modify and optimise the release profile of imiquimod from the nanoparticle.

In some favoured embodiments, the one or more lipid layers or bilayers may comprise lipids selected from the group consisting of cholesterol, phospholipids, lysolipids, lysophospholipids, and sphingolipids, and derivatives thereof. Suitable lipids include, but are not limited to, phosphatidylcholine (PC) (such as egg PC, soy PC), including 1,2-diacyl-glycero-3-phosphocholines; phosphatidylserine (PS); phosphatidylglycerol; phosphatidylinositol (PI); glycolipids; sphingophospholipids, such as sphingomyelin; sphingoglycolipids (also known as 1-ceramidyl glucosides), such as ceramide galactopyranoside, gangliosides and cerebrosides; fatty acids; sterols containing a carboxylic acid group such as cholesterol or derivatives thereof; and 1,2-diacyl-sn-glycero-3-phosphoethanolamines, including 1,2-dioleoyl-sn-glycero-3-phosphoethanolamine or 1,2-dioleolylglyceryl phosphatidylethanolamine (DOPE), 1,2-dihexadecylphosphoethanolamine (DHPE), 1,2-distearoylphosphatidylcholine (DSPC), 1,2-dipalmitoylphosphatidylcholine (DPPC), and 1,2˜dimyristoylphosphatidylcholine (DMPC). Suitable lipids also include natural lipids, such as tissue derived L-α-phosphatidyl: egg yolk, heart, brain, liver, soybean) and/or synthetic (e.g., saturated and unsaturated 1,2-diacyl-sn-glycero-3-phosphocholines, 1-acyl˜2-acyl-sn-glycero-3-phosphocholines, 1,2-diheptanoyl-SN-glycero-3-phosphocholine) derivatives of these lipids.

The outer lipid shell may also or alternatively comprise cationic lipids, including but not limited to N-[1-(2,3-dioleoyloxy)propyl]-N,N,N-trimethyl ammonium salts, also referred to as TAP lipids, for example as a methylsulfate salt. Suitable TAP lipids include, but are not limited to, DOTAP (dioleoyl-), DMTAP (dimyristoyl-), DPTAP (dipalmitoyl-), and DSTAP (distearoyl-). Other suitable cationic lipids include dimethyldioctadecyl ammonium bromide (DDAB), 1,2-diacyloxy-3-trimethylammonium propanes, N″[1-(2,3-dioloyloxy)propyl]-N,N-dimethyl amine (DODAP), 1,2-diacyloxy-3-dimethylammonium propanes, N-[1-(2,3-dioleyloxy)propyl]-N,N,N-trimethylammonium chloride (DOTMA), 1,2-dialkyloxy-3-dimethylammonium propanes, dioctadecylamidoglycylspermine (DOGS), 3-[N-(N′ 5N′-dimethylamino-ethane)carbamoyl]cholesterol (DC-Chol); 2,3-dioleoyloxy-N-(2-(sperminecarboxamido)-ethyl)-N,N-dimethyl-1-propanaminium trifluoro-acetate (DOSPA), β-alanyl cholesterol, cetyltrimethylammonium bromide (CTAB), diC₁₄-amidine, N-tert-butyl-N′-tetradecyl-3-tetradecylamino-propionamidine, N-(alpha-trimethylammonioacetyl)didodecyl-D-glutamate chloride (TMAG), ditetradecanoyl-N-(trimethylammonio-acetyl)diethanolamine chloride, 1,3-dioleoyloxy-2-(6-carboxy-spermyl)-propylamide (DOSPER), and N,N,N′N′-tetramethyl-N′-bis (2-hydroxylethyl)-2,3-dioleoyloxy-1,4-butanediammonium iodide, 1-[2-(acyloxy)ethyl]-2-alkyl(alkenyl)-3-(2-hydroxyethyl)-imidazolinium chloride derivatives, such as 1-[2-(9(Z)-octadecenoyloxy)ethyl]-2-(8(Z)-heptadecenyl-3-(2-hydroxyethyl)imidazolinium chloride (DOTIM) and 1-[2-(hexadecanoyloxy)ethyl]-2-pentadecyl-3-(2-hydroxyethyl)imidazolinium chloride (DPTIM), and 2,3-dialkyloxypropyl quaternary ammonium derivatives containing a hydroxyalkyl moiety on the quaternary amine, for example, 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimethyl-hydroxypropyl ammonium bromide (DORIE-HP), 1,2-dioleyl-oxy-propyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), and 1,2-disteryloxypropyl-3-dimethyl-hydroxyethyl ammonium bromide (DSRIE).

In some embodiments, the outer lipid shell may comprise a PEGylated derivative of a neutral, zwitterionic, cationic or anionic lipid, such as mPEG-DSPE, including DSPE PEG (2000 MW) and DSPE PEG (5000 MW). The surface display of PEG, or other suitable hydrophilic polyalkylene oxides, on the outer shell of the nanoparticle may serve to reduce uptake of the nanoparticle by the reticuloendothelial system (“RES”) when the nanoparticle is present in vivo; thereby prolonging in vivo residence and systemic circulation time, and/or allowing the nanoparticle to provide a sustained and prolonged immunostimulatory effect. Further examples of suitable PEGylated lipids include dipalmitoyl-glycero-succinate polyethylene glycol (DPGS-PEG), stearyl-polyethylene glycol and cholesteryl-polyethylene glycol.

In some embodiments, the outer lipid shell may comprise a mixture of phospholipids and cholesterol, such as a mixture of N-(carbonyl-) methoxypolyethylene glycol 2000)-1,2-distearoyl-sn-glycero 3-phosphoethanolamine sodium salt (mPEG-DSPE), phosphatidyl choline such as fully hydrogenated soy phosphatidylcholine (HSPC), and cholesterol. These lipids are well known and well characterised, being used in approved commercial products such as Doxil®. Alternative suitable phospholipids, known to the skilled person, may be used in place of the DSPE-PEG and/or HSPC. The lipids may be mixed and used in any desired molar ratio. For example, the molar ratio of the phospholipids to the cholesterol may range from about 1:1 to about 6:1, more preferably from about 1:1 to about 3:1, most preferably about 2:1. Where the phospholipids include DSPE-PEG and HSPC, these components may be present in a molar ratio DSPE-PEG:HSPC of about 1:1-1:200or 1:10-1:200; suitably from 1:1-1:100 or from 1:1-1:50 or from 1:1-1:30 or from 1:10-1:30; advantageously from 1:15-1:25. In some embodiments, the molar ratio of the HSPC: DSPE-PEG: cholesterol may be about 2:0.1:1 or about 2:0.01:1 or about 2:0.2:1.

The outer lipid shell of the nanoparticle encloses an inner aqueous core, which may comprise one or more hydrogel polymers which may serve to further stabilise and/or to control the release from the nanoparticle of the imiquimod and any other active agents which may be contained within the inner core of the nanoparticle, such as an antigen or polynucleotide as described in more detail below. The hydrogel polymers may be covalently and/or non-covalently cross-linked, or may be capable of being covalently and/or non-covalently cross-linked, or may have no cross-links. The hydrogel polymers may, for example, be or include poly(lactic acid), poly(glycolic acid), poly(lactic acid-co-glycolic acids), polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); poly(glycolide-co-caprolactones); polycarbonates; polyamides, polypeptides, and poly(amino acids); polyesteramides; other biocompatible polyesters; poly(dioxanones); poly (alkylene alkylates); hydrophilic polyethers; polyurethanes; polyetheresters; polyacetals; polycyanoacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), polyvinyl alcohols, polyvinylpyrrolidone; poly(alkylene oxides); celluloses, polyacrylic acids, albumin, collagen, gelatin, prolamines, and/or polysaccharides. In particular, the hydrogel polymers may include copolymers, including block copolymers, or blends of any of the aforementioned hydrogel polymers. In some embodiments, the inner aqueous core of the nanoparticle may comprise a polyethylene glycol polymer, such as polyethylene glycol 4000. PEG 4000 is widely used in pharmaceutical formulations, including parenteral formulations such as INVEGA SUSTENNA®. The inner aqueous core may additionally or alternatively comprise a block copolymer containing one or more poly(alkylene oxide) segments, such as polyethylene glycol, and one or more aliphatic polyester segments, such as polylactic acid.

The nanoparticle may have a diameter, measured by the standard, art-recognised technique of Dynamic Light Scattering (DLS), of no more than about 300 nm. In some embodiments, the diameter of the nanoparticle (measured by DLS) may be no more than about 200 nm, or no more than about 150 nm, or no more than about 130 nm, or no more than about 120 nm. The diameter of the nanoparticle (measured by DLS) may be at least 15 nm or at least 20 nm or at least 30 nm or at least 50 nm. Suitably, the diameter of the nanoparticle (measured by DLS) may be about 20-300 nm or about 20-150 nm or about 20-100 nm or about 20-50 nm or about 30-300 nm or about 50-150 nm or about 80-125 nm or about 90-110 nm. DLS may be performed according to ISO 22412:2017 or a similar technique.

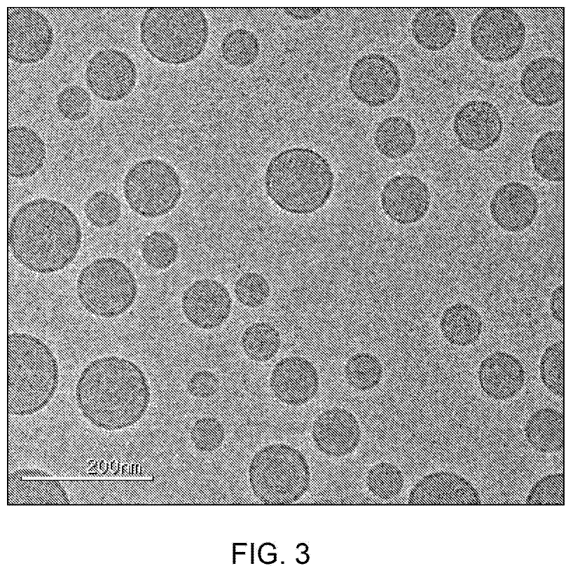

Advantageously, the nanoparticle may be spherical or spheroid, and/or may be unilamellar. Exemplary nanoparticles in accordance with the disclosure, viewed under Cryo transmission electron microscopy (cryo TEM) may be seen in FIG. 3. Here, it can be seen that the majority of the nanoparticles are spherical and unilamellar; and that the interior of the nanoparticles is more dense than the surrounding buffer, consistent with the loading of imiquimod, HP-β-CD and PEG 4000 polymer in the interior of the nanoparticle.

The inner aqueous core of the nanoparticle comprises imiquimod. Some or all of the imiquimod is complexed with a host molecule. As mentioned above, the term “imiquimod” as used herein embraces the imidazoquinoline known as 1-(2-methylpropyl)imidazo[4,5-c]quinolin-4-amine (CAS Number: 99011-02-6), R-837, and S-26308 and shown below:

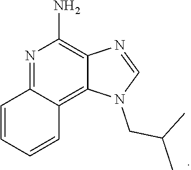 [SMILES: CC(C)Cn1cnc2c(N)nc3ccccc3c21]

The term further embraces structural analogues of imiquimod (R-837) as defined herein, which are active TLR7/8 ligands, including but not limited to resiquimod, gardiquimod, CL097, S28690, 852-A, 854A, CL075 and others known in the art and as defined above. Imiquimod as defined herein is a small synthetic guanosine analogue which is recognised for its immune-stimulating capabilities, and in particular is known to be effective in activating TLR7 and/or TLR8. In some preferred embodiments and aspects of this disclosure, the imiquimod is imiquimod (R-837).

Imiquimod (R-837) is approved for therapeutic administration as a cutaneous cream and is commercially available as a drug substance manufactured and tested in accordance with current Good Manufacturing Practices (cGMPs) under an active Drug Master File. Alternatively, imiquimod may be readily synthesised as a small molecule based on available raw materials and using methods well known in the art.

The inner aqueous core of the nanoparticle further comprises a host molecule that is capable of reversibly forming a complex, such as an inclusion complex, with imiquimod. An inclusion complex can be formed where an imiquimod molecule, or part of an imiquimod molecule, inserts into a cavity of a host molecule or group of host molecules. The host molecule may assist in solubilising the imiquimod in the aqueous core of the nanoparticle, and/or with controlling the release of the imiquimod from the nanoparticle. The imiquimod may, accordingly, be present in the form of an inclusion complex with the host molecule.

The host molecule may, for example, comprise a cyclodextrin; preferably a cyclodextrin selected from α-cyclodextrin; β-cyclodextrin; γ-cyclodextrin; methyl α-cyclodextrin; methyl β-cyclodextrin; methyl γ-cyclodextrin; ethyl β-cyclodextrin; butyl α-cyclodextrin; butyl β-cyclodextrin; butyl γ-cyclodextrin; pentyl γ-cyclodextrin; hydroxyethyl β-cyclodextrin; hydroxyethyl γ-cyclodextrin; 2-hydroxypropyl α-cyclodextrin; 2-hydroxypropyl β-cyclodextrin; 2-hydroxypropyl γ-cyclodextrin; 2-hydroxybutyl β-cyclodextrin; acetyl α-cyclodextrin; acetyl β-cyclodextrin; acetyl γ-cyclodextrin; propionyl β-cyclodextrin; butyryl β-cyclodextrin; succinyl α-cyclodextrin; succinyl β-cyclodextrin; succinyl γ-cyclodextrin; benzoyl β-cyclodextrin; palmityl β-cyclodextrin; toluenesulfonyl β-cyclodextrin; acetyl methyl β-cyclodextrin; acetyl butyl β-cyclodextrin; glucosyl α-cyclodextrin; glucosyl β-cyclodextrin; glucosyl γ-cyclodextrin; maltosyl α-cyclodextrin; maltosyl β-cyclodextrin; maltosyl γ-cyclodextrin; α-cyclodextrin carboxymethylether; β-cyclodextrin carboxymethylether; γ-cyclodextrin carboxymethylether; carboxymethylethyl β-cyclodextrin; phosphate ester α-cyclodextrin; phosphate ester β-cyclodextrin; phosphate ester γ-cyclodextrin; 3-trimethylammonium-2-hydroxypropyl β-cyclodextrin; sulfobutyl ether β-cyclodextrin; carboxymethyl α-cyclodextrin; carboxymethyl β-cyclodextrin; carboxymethyl γ-cyclodextrin, and combinations thereof. However, many other host molecules are known in the art and may be used in accordance with this disclosure, such as polysaccharides, cryptands, cryptophanes, cavitands, crown ethers, dendrimers, ion-exchange resins, calixarenes, valinomycins, nigericins, catenanes, polycatenanes, carcerands, cucurbiturils, and spherands, and others familiar to the skilled person.

In certain favoured embodiments, the host molecule is or includes 2-hydroxypropyl-β-cyclodextrin. 2-hydroxypropyl-β-cyclodextrin (HP-β-CD) (CAS number 128446-35-5) is a partially substituted poly (hydroxpropyl) ether of beta cyclodextrin (molar substitution 0.59-0.73 per anhydro glucose unit). It is capable of reversibly complexing with imiquimod, such as to improve the solubilisation of imiquimod in the aqueous inner core of the nanoparticle, whilst allowing for controlled release of imiquimod from the nanoparticle. HP-β-CD is currently used in a number of marketed products, including Mitozytrex™, a formulation of HP-β-CD and mitomycin approved for treatment of adenocarcinoma of the stomach or pancreas in the United States.

Preferred embodiments of this disclosure comprise imiquimod complexed with a host molecule. Some embodiments may also comprise uncomplexed imiquimod and/or host molecule within the inner aqueous core of the nanoparticles. In particular, the inner aqueous core of the nanoparticles may also comprise imiquimod dispersed, dissolved or suspended in the aqueous core, and/or imiquimod present in the form of a precipitate. Optionally, the nanoparticle may not include IL-2 and/or may not include a protein cytokine releasably attached to, associated with and/or encapsulated within the outer lipid shell.

The vaccine adjuvant of the present disclosure may be or may comprise an aqueous solution, aqueous dispersion or aqueous suspension of nanoparticles as disclosed, or may comprise a dried or lyophilised preparation that can be hydrated to produce an aqueous solution, dispersion or suspension of nanoparticles as disclosed. The adjuvant will typically be used in hydrated form, but may conveniently be prepared in lyophilised form, optionally for storage prior to use.

The vaccine adjuvant of the present disclosure may further comprise one or more additional adjuvant ingredients, such as some or all of the ingredients of an approved vaccine adjuvant. This may further improve the efficacy of the vaccine adjuvant for enhancing an immune response. The vaccine adjuvant of the present disclosure may, for example, comprise one or more additional adjuvant ingredients which are TLR agonists, including TLR4 agonists such as monophosphoryl lipid A (MPL), and/or TLR9 agonists such as CpG 1018. The vaccine adjuvant may, for example, further comprise MPL and QS-21, a commercially available saponin. The additional adjuvant ingredients may be present in the vaccine adjuvant in free form, and/or may be present in association with a delivery vehicle such as a liposome. Additionally or alternatively, the additional adjuvant ingredients may be loaded into or onto the vaccine adjuvant nanoparticles.

Also provided according to the present disclosure is a vaccine composition comprising (a) an antigen that is capable of inducing an immune response and/or a polynucleotide that encodes an antigen capable of inducing an immune response; and (b) a vaccine adjuvant comprising a plurality of nanoparticles in accordance with the present disclosure.

In some embodiments, some or all of the antigen and/or the polynucleotide of component (a) may be releasably attached to, associated with and/or encapsulated within the outer lipid shell of the nanoparticles of component (b). The antigen and/or polynucleotide may be encapsulated within the lipid shell of the nanoparticles of component (b), and/or may be dispersed within the aqueous core of the nanoparticles of component (b), and/or may be releasably attached to or associated with the lipid shell of the nanoparticles of component (b). Some or all of the antigen and/or polynucleotide may, optionally, be reversibly associated with a host molecule within the aqueous core of the nanoparticles of component (b). In some embodiments, the antigen and/or polynucleotide may be non-covalently attached to the lipid shell of the nanoparticles of component (b), for example by ionic interaction, by hydrogen bonding, or by Van der Waals interactions. In some other embodiments, the antigen and/or polynucleotide may be covalently attached to the lipid shell of the nanoparticles of component (b) by way of a cleavable linking group, which linking group can be cleaved under suitable conditions, such as at a certain ambient pH or in the presence of certain cleaving agent(s), such as to release the antigen and/or polynucleotide.

Component (a) may, additionally or alternatively, comprise a delivery vehicle which is loaded with the antigen and/or polynucleotide, and which is capable of releasing the antigen and/or polynucleotide in vivo. The delivery vehicle may, for example, be a nanoparticulate vehicle such as a polymeric nanoparticle, a liposome, or a nanoparticle comprising an outer lipid shell and an inner aqueous core encapsulated within the outer lipid shell, as herein described. The delivery vehicle may, for example, be a nanogel or a PLGA nanoparticle, as described in Look et al, Biomaterials 35 (2014) 1089-1095. Some or all of the antigen and/or polynucleotide may be releasably attached to, associated with and/or encapsulated within the nanoparticulate delivery vehicle. Where the nanoparticulate delivery vehicle comprises a core/shell nanoparticle of the type herein described, some or all of the antigen and/or polynucleotide may be releasably attached to, associated with and/or encapsulated within the outer lipid shell of the nanoparticle. The outer lipid shell of this nanoparticle may comprise one or more lipid layers or bilayers enclosing a central core, as herein described. The inner aqueous core of the nanoparticle may comprise a hydrogel, as herein described. The inner aqueous core of the nanoparticle may further comprise a host molecule, as herein described.

In some embodiments of the vaccine composition, component (a) may comprise a polynucleotide which is a DNA molecule or an RNA molecule, such as an mRNA molecule or siRNA molecule. DNA and RNA vaccines are known in the art. These known vaccines contain DNA or RNA polynucleotides which are capable of being expressed in vivo to yield an antigen that can stimulate a protective or therapeutic immune response. Recent examples include mRNA vaccines which have been developed for protection against SARS-CoV-2 infection and COVID-19 disease. DNA vaccines for protection against and treatment of cancer have also been described in the art. Component (a) of the vaccine composition may accordingly comprise a DNA or RNA molecule, such as an mRNA molecule, which is capable of expressing an antigen that can stimulate a protective or therapeutic immune response in vivo.

In some embodiments, component (a) of the vaccine composition may comprise a viral antigen, and/or a bacterial antigen, and/or a fungal antigen, and/or a disease-associated and/or cancer-associated antigen; and/or may comprise a polynucleotide which encodes a viral antigen, and/or a bacterial antigen, and/or a fungal antigen, and/or a disease-associated and/or cancer-associated antigen. Component (a) may comprise an antigen which is a peptide, protein, carbohydrate, nucleic acid, and/or lipid molecule or structure. Component (a) may comprise a polynucleotide which encodes a peptide antigen or a protein antigen.

In some embodiments, component (a) of the vaccine composition may comprise a coronavirus or coronavirus-associated antigen, such a SARS-CoV, MERS-CoV or SARS-CoV-2 antigen; or an influenza or influenza-associated antigen, such as an influenza A, influenza B, influenza C or influenza D antigen; or a Herpes simplex (HSV-1 or HSV-2) or HSV-associated antigen; or a cytomegalovirus (CMV) or CMV-associated antigen; or a Lyme's Disease (Borrelia) or Lyme's Disease-associated antigen; or a Respiratory Syncytial Virus (RSV) or RSV-associated antigen; or an Epstein-Barr Virus (EBV) or EBV-associated antigen; or a Zika virus or Zika virus-associated antigen; or a meningitis or meningitis-associated antigen; or a measles or measles-associated antigen; or a mumps or mumps-associated antigen; or a rubella or rubella-associated antigen; or a varicella (chickenpox) or chickenpox-associated antigen; or a Herpes zoster (shingles) or shingles-associated antigen; or a diphtheria or diphetheria-associated antigen; or a tetanus or tetanus-associated antigen; or a poliomyelitis or poliomyelitis-associated antigen; or a dengue virus or dengue virus-associated antigen; or a Haemophilus influenzae (Hib) or Hib-associated antigen; or a rotavirus or rotavirus-associated antigen; or a Streptococcus pneumoniae (Streptococcus) or Streptococcus-associated antigen; or a human papillomavirus (HPV) or HPV-associated antigen; or a pertussis or pertussis-associated antigen; or a hepatitis or hepatitis-associated antigen; or a tuberculosis or tuberculosis-associated antigen; or a human immunodeficiency virus (HIV) or HIV-associated antigen; or an adenovirus or adenovirus-associated antigen; or an anthrax or anthrax-associated antigen; or a cholera or cholera-associated antigen; or a Japanese Encephalitis (JE) or JE-associated antigen; or a rabies or rabies-associated antigen; or a smallpox or smallpox-associated antigen; or a Typhoid Fever (typhoid) or typhoid-associated antigen; or a yellow fever or yellow fever-associated antigen; or an Ebola or Ebola-associated antigen; or a cancer or cancer-associated antigen.

In some embodiments, component (a) of the vaccine composition may comprise a polynucleotide which encodes a coronavirus or coronavirus-associated antigen, such a SARS-CoV, MERS-CoV or SARS-CoV-2 antigen; or an influenza or influenza-associated antigen, such as an influenza A, influenza B, influenza C or influenza D antigen; or a Herpes simplex (HSV-1 or HSV-2) or HSV-associated antigen; or a cytomegalovirus (CMV) or CMV-associated antigen; or a Lyme's Disease (Borrelia) or Lyme's Disease-associated antigen; or a Respiratory Syncytial Virus (RSV) or RSV-associated antigen; or an Epstein-Barr Virus (EBV) or EBV-associated antigen; or a Zika virus or Zika virus-associated antigen; or a meningitis or meningitis-associated antigen; or a measles or measles-associated antigen; or a mumps or mumps-associated antigen; or a rubella or rubella-associated antigen; or a varicella (chickenpox) or chickenpox-associated antigen; or a Herpes zoster (shingles) or shingles-associated antigen; or a diphtheria or diphetheria-associated antigen; or a tetanus or tetanus-associated antigen; or a poliomyelitis or poliomyelitis-associated antigen; or a dengue virus or dengue virus-associated antigen; or a Haemophilus influenzae (Hib) or Hib-associated antigen; or a rotavirus or rotavirus-associated antigen; or a Streptococcus pneumoniae (Streptococcus) or Streptococcus-associated antigen; or a human papillomavirus (HPV) or HPV-associated antigen; or a pertussis or pertussis-associated antigen; or a hepatitis or hepatitis-associated antigen; or a tuberculosis or tuberculosis-associated antigen; or a human immunodeficiency virus (HIV) or HIV-associated antigen; or an adenovirus or adenovirus-associated antigen; or an anthrax or anthrax-associated antigen; or a cholera or cholera-associated antigen; or a Japanese Encephalitis (JE) or JE-associated antigen; or a rabies or rabies-associated antigen; or a smallpox or smallpox-associated antigen; or a Typhoid Fever (typhoid) or typhoid-associated antigen; or a yellow fever or yellow fever-associated antigen; or an Ebola or Ebola-associated antigen; or a cancer or cancer-associated antigen.

The vaccine composition may be or may comprise an aqueous solution, aqueous dispersion or aqueous suspension. Alternatively, the vaccine composition may be provided in dried or lyophilised form. The vaccine composition may optionally include one or more additional excipients and/or active ingredients; such as stabilisers, preservatives, emulsifiers, buffering agents and/or additional adjuvants; including, without limitation, aluminium or aluminium salts, MF59 (squalene oil), thiomersal, gelatine, sorbitol, lipids (including 4-hydroxybutyl)azanediyl)bis(hexane-6,1-diyl)bis(2-hexyldecanoate), 2 [(polyethylene glycol)-2000]-N,N-ditetradecylacetamide, 1,2-Distearoyl-sn-glycero-3-phosphocholine, and cholesterol), potassium chloride, monobasic potassium phosphate, sodium chloride, dibasic sodium phosphate dihydrate, sucrose, tromethamine and tromethamine hydrochloride. Some or all of the antigen and/or the polynucleotide and/or the vaccine adjuvant and/or additional ingredients may be mixed, dissolved, dispersed or suspended in the vaccine composition.

The vaccine composition may, in some embodiments, comprise some or all of the active and/or excipient ingredients of a vaccine formulation which has been developed for prophylactic or therapeutic use; such as an approved vaccine formulation. Conveniently, the approved vaccine formulation may comprise an antigen capable of inducing an immune response and/or a polynucleotide that encodes an antigen capable of inducing an immune response. In these embodiments, the vaccine composition of the present disclosure may comprise the ingredients of the approved vaccine formulation mixed or otherwise formulated with a vaccine adjuvant comprising nanoparticles in accordance with the present disclosure. The approved vaccine formulation may, for example, be an approved vaccine against coronavirus, such as SARS-CoV, MERS-CoV or SARS-CoV-2, and/or an approved vaccine against influenza, and/or an approved vaccine against Herpes simplex (HSV-1 or HSV-2), and/or an approved vaccine against cytomegalovirus (CMV), and/or an approved vaccine against Lyme's Disease (Borrelia), and/or an approved vaccine against Respiratory Syncytial Virus (RSV), and/or an approved vaccine against Epstein-Barr Virus (EBV), and/or an approved vaccine against Zika virus, and/or an approved vaccine against meningitis, and/or an approved vaccine against measles, and/or an approved vaccine against mumps, and/or an approved vaccine against rubella, and/or an approved vaccine against varicella (chickenpox), and/or an approved vaccine against Herpes zoster (shingles), and/or an approved vaccine against diphtheria, and/or an approved vaccine against tetanus, and/or an approved vaccine against poliomyelitis, and/or an approved vaccine against dengue virus, and/or an approved vaccine against Haemophilus influenzae (Hib), and. or against rotavirus, and/or an approved vaccine against Streptococcus pneumonia, and/or an approved vaccine against human papillomavirus (HPV), and/or an approved vaccine against pertussis, and/or an approved vaccine against hepatitis, and/or an approved vaccine against tuberculosis, and/or an approved vaccine against human immunodeficiency virus (HIV), and/or an approved vaccine against adenovirus, and/or an approved vaccine against anthrax, and/or an approved vaccine against cholera, and/or an approved vaccine against Japanese Encephalitis (JE), and/or an approved vaccine against rabies, and/or an approved vaccine against smallpox, and/or an approved vaccine against Typhoid Fever, and/or an approved vaccine against yellow fever, and/or an approved vaccine against Ebola, and/or an approved vaccine against cancer.

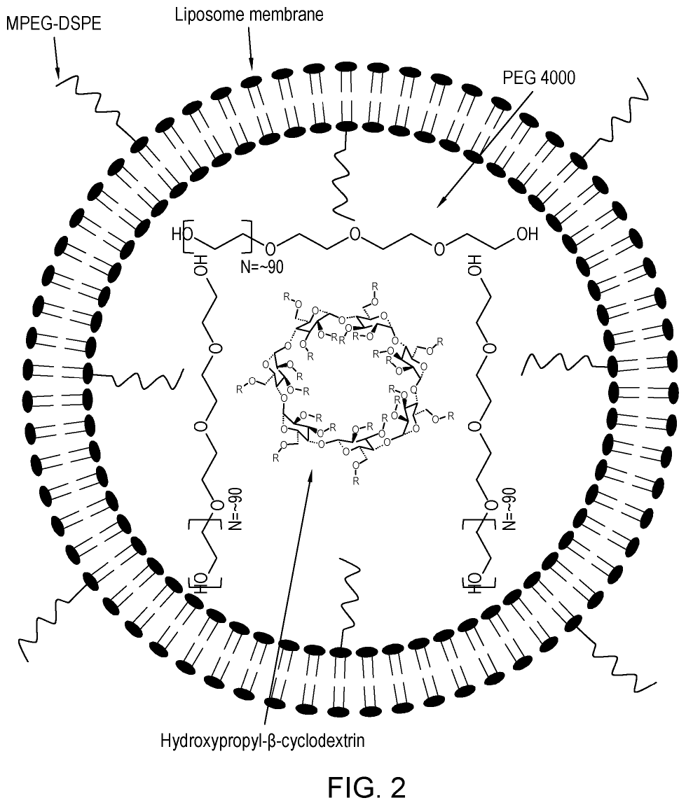

An exemplary nanoparticle in accordance with the present disclosure is illustrated in FIG. 2. As shown in this Figure, the exemplary nanoparticle has an outer liposomal shell formed from a lipid bilayer which includes mPEG-DSPE, where mPEG chains are displayed on the outer and inner surfaces of the liposomal shell. The inner aqueous core of the nanoparticle comprises a PEG 4000 hydrogel and hydroxypropyl-β-cyclodextrin host molecules. The host molecules reversibly associate with imiquimod molecules in the inner aqueous core of the nanoparticle (not shown). In some embodiments, as herein disclosed, an antigen or polynucleotide in accordance with this disclosure may be loaded into the nanoparticle; and may be reversibly attached to or within the outer liposomal shell of the nanoparticle and/or disposed wholly or partly within the inner core of the nanoparticle (not shown).

The vaccine adjuvants and/or the vaccine compositions of the present disclosure are preferably suitable for administration to a human or animal for prophylactic or therapeutic purposes. The adjuvants and/or compositions may be suitable for parenteral administration, particularly by injection, infusion or deposition. The adjuvants and/or compositions are preferably sterile. The nanoparticles contained in the vaccine adjuvants and vaccine compositions are preferably biodegradable.

The adjuvants and/or compositions may be buffered to a pH of at least about 6.5, or at least about 7; and preferably no more than pH 9 or no more than pH 8.5. Suitably, the adjuvants and/or compositions may be buffered to a pH which is suitably between about pH 6.5-9 or between about pH 6.5-8.5 or between about pH 6.5-8 or between about pH 7-9 or between about pH 7-8.5 or between about pH 7-8 or between about pH 7.5-9. This is beneficial and desirable when it comes to therapeutic use, where the adjuvant or composition should ideally be buffered to a pH which is close to physiological pH (pH 7.4). Buffering of the adjuvants or compositions may be effected using any suitable and acceptable buffering agents, such as citric acid/sodium citrate.

The vaccine adjuvant and/or the vaccine composition of the present disclosure may, suitably, be substantially free of unencapsulated imiquimod. This will help to avoid unwanted precipitation of imiquimod in or from the adjuvants or compositions.

The vaccine adjuvant and/or the vaccine composition may suitably comprise about 0.01-50 μg/ml of imiquimod. In some embodiments, the adjuvant or composition may comprise about 1-30 μg/ml of imiquimod or about 5-25 μg/ml of imiquimod or about 5-15 μg/ml of imiquimod. The adjuvant or composition may comprise at least about 1 μg/ml of imiquimod, or at least about 2 μg/ml of imiquimod, or at least about 3μg/ml of imiquimod, or at least about 5 μg/ml of imiquimod. The adjuvant or composition may comprise no more than about 20 μg/ml of imiquimod or no more than about 15 μg/ml of imiquimod or no more than about 12 μg/ml of imiquimod. The adjuvant or composition may comprise about 5 μg/ml, or about 10 μg/ml, or about 15 μg/ml of imiquimod.

The quantity or concentration of antigen or polynucleotide in the vaccine composition may be determined by the skilled person, taking into account usual considerations including the strength of the required response, the immunogenicity of the antigen, toxicity issues, and other criteria familiar to the skilled person.

The vaccine adjuvant and/or the vaccine composition may suitably comprise about 1-100 mg/ml of lipids. In some embodiments, the adjuvant or composition may comprise about 5-50 mg/ml of lipids or about 10-40 mg/ml of lipids or about 20-30 mg/ml of lipids. The adjuvant or composition may comprise at least about 5 mg/ml of lipids or at least about 10 mg/ml of lipids or at least about 15 mg/ml of lipids or at least about 20 mg/ml of lipids. The adjuvant or composition may comprise no more than about 50 mg/ml of lipids or no more than about 40 mg/ml of lipids or no more than about 30 mg/ml of lipids or no more than about 25 mg/ml of lipids.

The average diameter of the nanoparticles in the adjuvant or composition, measured by the standard, art-recognised technique of Dynamic Light Scattering (DLS), preferably according to ISO 22412: 2017, may suitably be no more than about 300 nm, or no more than about 200 nm, or no more than about 150 nm, or no more than about 130 nm, or no more than about 120 nm. Here, the average size of the nanoparticles in the adjuvant or composition may refer to the mean diameter of nanoparticles in the adjuvant or composition, or may refer to the median particle diameter D50 of the nanoparticles in the adjuvant or composition. In some embodiments, the average diameter of the nanoparticles in the adjuvant or composition may be at least about 15 nm or at least about 20 nm or at least about 30nm or at least about 50 nm. In some embodiments, the average diameter of the nanoparticles in the adjuvant or composition and/or the size range of the nanoparticles in the adjuvant or composition may be about 20-300 nm or about 20-150 nm or about 20-100 nm or about 20-50 nm or about 30-300 nm or about 50-150 nm or about 80-125 nm or about 90-110 nm.

As described in more detail below, the vaccine adjuvants and compositions of the present disclosure can provide for sustained release and/or delivery of imiquimod to a subject over an extended period of time, which allows for effective enhancement of a vaccine-induced immune response. Imiquimod is known in the art as a vaccine adjuvant that is capable of enhancing the immune response induced by vaccine antigens, including infectious disease or pathogen antigens and cancer-associated antigens. The present disclosure provides an approach and platform which permits imiquimod to be co-administered by injection together with existing approved adjuvant and/or vaccine ingredients for combined adjuvant and/or immunogenic action, where the mode and manner of administration and delivery may enhance the efficacy of the vaccine, reduce systemic exposure and associated toxicities, improve the pharmacokinetics, and/or provide prophylactic and therapeutic benefit at doses which are substantially below the approved therapeutic dose of imiquimod.

The vaccine adjuvants of the present disclosure may be suitable for enhancing the protective or therapeutic immune response induced by vaccines against a variety of different diseases and medical conditions, including viral, bacterial or fungal diseases, colonisations or infections and proliferative disorders including cancers. Another aspect of the present disclosure accordingly provides a method of enhancing an immune response to a vaccine in a subject, such as a human subject, comprising the step of administering to the subject a vaccine adjuvant as herein disclosed, where the vaccine adjuvant is administered to the subject before, simultaneously, and/or after administration of the vaccine.

The vaccine compositions of the present disclosure may be suitable for inducing an immune response in a subject which is effective to prevent or treat a variety of different diseases or medical conditions, including viral, bacterial or fungal diseases, colonisations or infections and proliferative disorders including cancers. Another aspect of the present disclosure accordingly provides a method for inducing a protective or therapeutic immune response in a subject, such as a human subject, and/or for immunising a subject, such as a human subject, against a viral, bacterial or fungal disease or colonisation or injection or against a proliferative disorder such as a cancer, comprising the step of administering to the subject a vaccine composition as herein disclosed.

The vaccine adjuvant and/or vaccine composition may be administered to the subject parenterally, for example by intravenous, intramuscular or subcutaneous injection or infusion or deposition. The vaccine adjuvant or vaccine composition may be administered orally, intranasally, intramuscularly, intradermally, transdermally, intravenously, peritoneally, intrathecally, intravesically, cutaneously, subcutaneously, or ocularly, including subconjunctivally, retrobulbarly, intracamerally, and intravitreally. The vaccine adjuvant or vaccine composition may be administered systemically, for example by intravenous injection or infusion; or may be administered locally, for example by injection into a lesion or the immediate locality of a lesion, such as a tumor. Local administration may be particularly relevant for immunisation against or treatment of cancer. In such cases, localised administration of the vaccine composition or vaccine adjuvant may have certain advantages over systemic administration. Notably, localised administration means that systemic exposure to the vaccine or adjuvant is minimised, the RES and tumor vasculature barriers are by-passed, and local/regional spread of the cancer can be more effectively addressed.

The vaccine adjuvant or vaccine composition may be administered as a single dose or as a plurality of doses. Each dose may suitably comprise about 1 ng-100 μg of imiquimod; suitably at least about 1 ng or at least about 5 ng or at least about 10 ng or at least about 50 ng or at least about 100 ng or at least about 500 ng or at least about 1 μg or at least about 5 μg or at least about 10 μg of imiquimod per dose. Additionally or alternatively, each dose may suitably comprise no more than about 100 μg or no more than about 75 μg or no more than about 50 μg or no more than about 25 μg or no more than about 20 μg or no more than about 10 μg or no more than about 5 μg or no more than about 1 μg of imiquimod. Each dose may suitably comprise about 1-100 ng or about 100 ng-1 μg or about 1-10 μg or about 10-100 μg of imiquimod.

Further embraced within the scope of this disclosure are vaccine adjuvants that are suitable for and/or are provided for use in enhancing an immune response to a vaccine in a subject; and vaccine compositions that are suitable for and/or are provided for use in inducing an immune response in a subject. Also provided is a vaccine adjuvant in accordance with the present disclosure, for use in manufacturing a composition for use in enhancing an immune response to a vaccine in a subject. Also provided is a vaccine composition in accordance with the present disclosure, for use in manufacturing a composition for use in inducing an immune response in a subject.

The present disclosure further provides a method for manufacturing a vaccine adjuvant as disclosed herein, comprising the sequential steps of:
(a) solubilising imiquimod with the host molecule in aqueous solution at a pH buffered to about pH 6 or below; preferably to about pH 4-6, or to about pH 4.5-6, or to about pH 4-5.5, or to about pH 5-6;
(b) combining the resulting aqueous solution with lipids to form lipid shell nanoparticles encapsulating imiquimod; and
(c) increasing the buffered pH of the formulation to about pH 6.5 or above, or to pH 7 or above, or to about pH 6.5-9 or to about pH 6.5-8.5, or to about pH 6.5-8; or to about pH 7-9 or to about pH 7-8.5 or to about pH 7-8 or to about pH 7.5-9.

This method allows for adjustment of the pH during the process in such a manner as to achieve a formulation at neutral (physiological) pH, whilst minimising or avoiding precipitation of unencapsulated imiquimod from the formulation. The method is compatible with the addition of antigen or polynucleotide, as described herein.

Suitably, step (a) of the process may involve solubilising imiquimod at a pH buffered to about pH 4-6, or to about pH 4.5-6, or to about pH 4-5.5, or to about pH 5-6. Step (a) may, for example, involve solubilising imiquimod in aqueous solution in the presence of a hydroxyacid, such as citric acid, tartaric acid, lactic acid, glycolic acid or malic acid. Step (a) may involve solubilising imiquimod in aqueous solution in the presence of a host molecule as disclosed herein, such as a cyclodextrin, in particular HP-β-CD. In particular, step (a) may involve combining imiquimod with a host molecule such as cyclodextrin in solution at a pH buffered to about pH 6 or below, preferably to about pH 4-6, or to about pH 4.5-6, or to about pH 4-5.5, or to about pH 3-5.5, or to about pH 5-6, or to about pH 5.

Step (b) may involve mixing the solution of (a) with lipids as disclosed herein, to form a solution or suspension of multilamellar structures; and processing these multilamellar structures to form lipid shell nanoparticles encapsulating imiquimod. The lipids may optionally be solubilised in alcoholic solution. The step of processing the structures may comprise extruding the solution or suspension of multilamellar structures through membranes to form lipid shell nanoparticles encapsulating imiquimod; or may comprise drying the solution or suspension of multilamellar structures to form a film, solubilising the film, and shaking or sonicating the resulting solution to form lipid shell nanoparticles encapsulating imiquimod; or may comprise using microfluidic mixing technologies to form lipid shell nanoparticles encapsulating imiquimod.

In other embodiments, step (b) may involve mixing the solution of (a) with empty liposomes to form lipid shell nanoparticles encapsulating imiquimod. In this case, the empty liposomes may be formed from lipids as disclosed herein.

Step (c) of the process may involve increasing the buffered pH of the formulation to about pH 6.5 or above, or to pH 7 or above, or to about pH 6.5-9 or to about pH 6.5-8.5, or to about pH 6.5-8; or to about pH 7-9 or to about pH 7-8.5 or to about pH 7-8 or to about pH 7.5-9. In some embodiments, the buffered pH of the formulation may be increased in step (c) by known techniques such as diafiltration or buffer exchange.

The method may optionally further comprise a step of adding a hydrogel polymer as disclosed herein, such as a PEG 4000 polymer, after step (a). Adding the hydrogel polymer in this way allows for the hydrogel polymer to be incorporated into the aqueous core of the nanoparticles. Optionally, the method may not include a step of adding IL-2 or a protein cytokine such as IL-2 to the formulation of (c) to load the nanoparticles with the protein cytokine.

In some embodiments, the method may further comprise reducing unencapsulated imiquimod from the nanoparticle formulation of (b), after step (b) and before step (c). This may optionally be done by ultracentrifugation or by diafiltration with membranes which are sized to retain the nanoparticles but permit the passage of free imiquimod, or by other techniques known in the art.

The method may further comprise standard processing steps, including concentration adjustment, the addition of suitable excipients, and sterilisation. In particular, the method may comprise a step of adding one or more additional adjuvant ingredients as herein disclosed. The one or more additional adjuvant ingredients may be added during or after step (a). In some embodiments, one or more or all of the additional ingredients may be added after step (c), after the buffered pH of the formulation has been increased.

Once produced, the vaccine adjuvant may optionally be dried or lyophilised, and/or may be dispensed into a container for storage or administration. Alternatively, the vaccine adjuvant may be further processed to provide a vaccine composition in accordance with the disclosure, as further described below.

The present disclosure further provides a method for producing a vaccine composition as disclosed herein, which comprises providing a vaccine adjuvant as disclosed herein and adding an antigen that is capable of inducing an immune response and/or a polynucleotide that encodes an antigen capable of inducing an immune response. The step of adding an antigen or polynucleotide may, for example, comprise adding one or more ingredients of an approved vaccine; and may, in particular, comprise adding an approved vaccine formulation.

In some embodiments, the method may comprise manufacturing a vaccine adjuvant according to the methods disclosed herein and adding an antigen that is capable of inducing an immune response and/or a polynucleotide that encodes an antigen capable of inducing an immune response. The antigen and/or polynucleotide may be added during or after step (a). In particular, the antigen and/or polynucleotide may be added during step (a), or between step (a) and step (b), or during step (b), or between step (b) and step (c), or during step (c), or after step (c). In some embodiments, the method may comprise adding the polynucleotide during step (a), between step (a) and step (b), or during step (b). Where the antigen is a small molecule, or is a substance or material which is not impaired by acidic pH, the method may comprise adding the antigen during step (a), between step (a) and step (b), or during step (b). Where the antigen is or includes a large molecule, and/or is a substance or material which is impaired by acidic pH, the method may comprise adding the antigen after step (c). Being “impaired” in this context includes any change which materially affects the immunogenic properties of the antigen.

In other embodiments, the method may comprise providing a vaccine adjuvant as disclosed herein and combining the vaccine adjuvant with an antigen that is capable of inducing an immune response and/or a polynucleotide that encodes an antigen capable of inducing an immune response. The step of providing the vaccine adjuvant may, optionally, comprise manufacturing the vaccine adjuvant according to the methods disclosed herein. Alternatively, the step of providing the vaccine adjuvant may comprise obtaining a previously manufactured vaccine adjuvant. The step of combining the vaccine adjuvant with the antigen and/or polynucleotide may comprise mixing the vaccine adjuvant with the antigen and/or polynucleotide. It will be appreciated that this may involve adding the vaccine adjuvant to the antigen and/or polynucleotide, or adding the antigen and/or polynucleotide to the vaccine adjuvant.

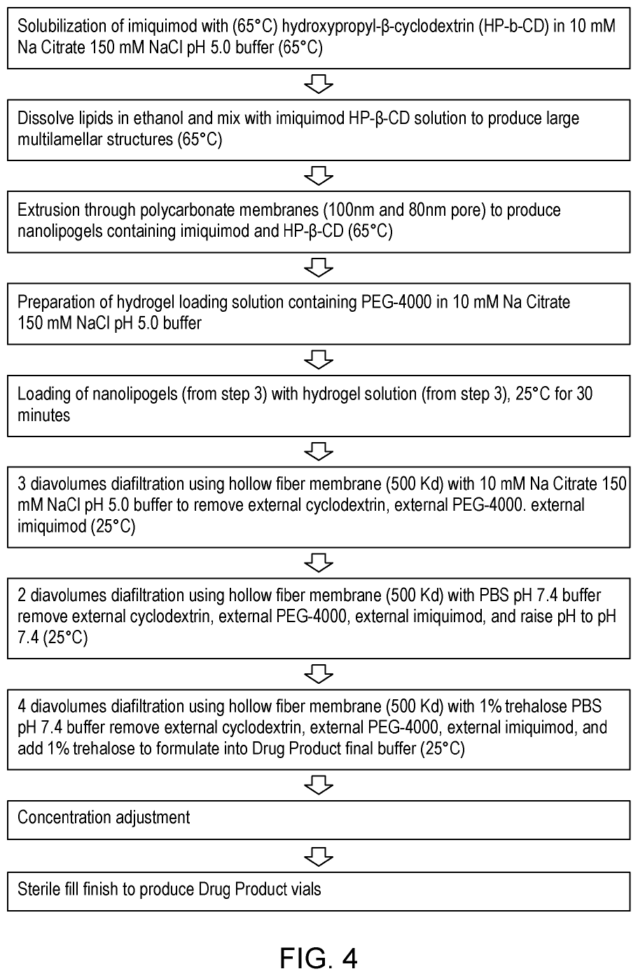

An example of the manufacturing process is illustrated in FIG. 4 and described in Examples 1-3. The process as illustrated and described utilizes acidic pH buffers and HP-β-CD concentrations which are optimized for the incorporation of imiquimod:cyclodextrin into nanoparticles during the extrusion process. The present inventors have recognised that as acidic pH conditions are not compatible with lipid and antigen/polynucleotide stability, and are unsuitable for parenterally administered drugs, the product ingredients should not be exposed to a pH below pH 4 during the manufacturing process, and the pH must be raised to near neutral pH in the final product. This may or may not be before the addition of any antigen or polynucleotide. Because imiquimod solubility is greatly decreased at near neutral pH, the concentration of “free” unencapsulated imiquimod in the in-process solution must be reduced prior to any increase in pH to prevent precipitation of the “free” unencapsulated imiquimod which would block filters used for dialfiltration and sterile filtration, preventing its removal from the bulk product and preventing successful sterile filtration. The reduction in the concentration of “free” unencapsulated imiquimod can be accomplished by methods such as ultracentrifugation or diafiltration with appropriately sized membranes that retain nanoparticles, but permit the passage of free imiquimod and cyclodextrin into the permeate. Diafiltration is a process that is well suited for clinical and commercial scale drug manufacturing, and may be preferred for this reason. After the concentration of “free” unencapsulated imiquimod in the in-process solution is reduced sufficiently so that it does not precipitate at near neutral pH, the pH can be raised and diafiltration can continue to remove external cyclodextrin and imiquimod, and formulate the product into Drug Product final buffer.

The vaccine adjuvant may be mixed with antigen or polynucleotide for production of a vaccine composition. This step may be carried out during or after the production of the vaccine adjuvant. Where a polynucleotide is to be added, it may be considered favourable to add the polynucleotide during the early stages of production of the adjuvant. Where a larger protein antigen or cellular antigen is to be added, it may be considered favourable to add the larger protein antigen or cellular antigen during or after the later stages of production of the adjuvant, once the pH has been adjusted to near neutral, as disclosed herein. This may be before or after all necessary diafiltration and purification steps have been completed, as described in Example 2.

A batch process performed according to cGMP standards may produce approximately 10 liters of vaccine adjuvant, in the form of a sterile suspension of liposomes containing imiquimod at a concentration of approximately 10 μg/ml. This composition may be used as an adjuvant in a vaccine, by combination with other vaccine ingredients including antigens or polynucleotides which encode antigens.

Following are specific examples according to the present disclosure.

### Example 1: Nanoparticle Components

Nanoparticles were produced using a number of raw materials and excipients important for the structure and quality of the drug product. With the exception of cholesterol (see below), all of the raw materials and excipients were either synthetic or derived from plants.

The liposome shell was composed of three components:
N-(carbonyl-) methoxypolyethylene glycol 2000)-1,2-distearoyl-sn-glycero 3-phosphoethanolamine sodium salt (MPEG-DSPE)
fully hydrogenated soy phosphatidylcholine (HSPC), and
cholesterol

The aqueous core was composed of two components:
2-hydroxypropyl-β-cyclodextrin
polyethylene glycol 4000 hydrogel

These components are all commercially available and are known and well-characterised excipients.

### Example 2: Preparation of a Nanoparticulate Vaccine Adjuvant Comprising Imiquimod

The steps of a process for production of nanoparticles loaded with imiquimod is illustrated in FIG. 4. Imiquimod was solubilised in aqueous solution at pH 5 in the presence of HP-β-CD. A solution of lipids was produced by dissolving fully hydrogenated soy phosphatidylcholine (HSPC), N-(carbonyl-methoxypolyethylene glycol 2000)-1,2-distearoyl-sn-glycero 3-phosphoethanolamine sodium salt (MPEG-DSPE) and cholesterol in ethanol at a molar ratio of about 1.9:0.1:1). This solution was mixed with the imiquimod solution to form multilamellar structures. These structures were then extruded through membranes with appropriate pore sizes (80 to 100 nm) to produce nanoparticles or “nanolipogels”, which are core-shell structures having a lipid shell encapsulating imiquimod and HP-β-CD.

The nanolipogels were incubated with a solution of PEG-4000 in pH 5 buffer, allowing the PEG-4000 to load into the interior of the nanoparticles.

Diafiltration was carried out to remove external imiquimod and cyclodextrin. Further diafiltration steps were then performed to raise the pH of the formulation to pH 7.4. The resulting solution is suitable for use as a vaccine adjuvant.

### Example 3: Preparation of a Vaccine Composition

To prepare a vaccine composition, the nanoparticles of Example 2 are mixed with vaccine ingredients, including a suitable vaccine antigen such as SARS-CoV-2 spike protein, in solution at pH 7.4. SARS-CoV-2 spike protein is utilised as an antigen in various approved vaccine formulations, including the NUVAXOVID® SARS-CoV-2 vaccine, and is commercially available.

The nanoparticles are further subjected to 4 diavolumes diafiltration using hollow fiber membrane (500 Kd) with 1% trehalose PBS pH 7.4 buffer at 25° C., to further reduce external cyclodextrin concentration and remove external imiquimod, and to add 1% trehalose. This step may, alternatively, be carried out during the production of the nanoparticles according to Example 2.

The concentration of the formulation is then adjusted and the formulation was sterilised to a standard required for therapeutic use. Sterilisation is carried out by filtration, using a 0.2 μm filter.

The formulation is supplied at a single dose concentration containing 10 μg/mL of imiquimod. It is a sterile white opaque liquid. The container closure system consists of a 5 mL clear borosilicate glass vial, a 13 mm synthetic chlorobutyl rubber stopper, and flip-off crimp seal. The composition of the IMP in a 5 ml vial is listed in Table 2 below.

TABLE 2

| Component | Amount (mg) | Function | Quality Standard |
|---|---|---|---|
| Imiquimod | 0.05 | Active Ingredient | USP |
| SARS-CoV-2 spike | 0.05 | Active Ingredient | Manufacturer |
| protein | | | specifications |
| HSPC | 69.3 | Vesicle forming | Manufacturer |
| | | lipid | specifications |
| MPEG-DSPE | 12.6 | Vesicle forming | Manufacturer |
| | | lipid | specifications |
| Cholesterol | 17.7 | Vesicle forming | Multi (USP/Ph. |
| | | lipid | Eur./BP/JP) |
| PEG 4000 | 39* | Drug trapping agent | Manufacturer |
| | | | specifications |
| 2-hydroxypropyl-β- | 1170* | Drug trapping agent | USP/Ph. Eur. |
| cyclodextrin | | | |
| Sodium phosphate | 13 | Buffer | Multi (USP/Ph. |
| | | | Eur./BP/JP) |
| Sodium chloride | 17 | Isotonicity | Multi (USP/Ph. |
| | | | Eur./BP/JP) |
| Trehalose | 18 | Isotonicity | Multi (USP/Ph. |
| | | | Eur./BP/JP) |
| Water for injection | 5,000 | Solvent | Multi (USP/Ph. Eur./JP) |
| Sodium hydroxide | as required | pH adjustor | Multi (USP/Ph. |
| | | | Eur./BP/JP) |
| Hydrochloric acid | as required | pH adjustor | NF/Multi (USP/Ph. |
| | | | Eur./BP/JP) |

*The concentrations of the PEG 4000 and 2-hydroxypropyl-β-cyclodextrin are the maximum possible concentrations based upon the batch formula.

### Example 4: Drug Product Properties

A Cryo-TEM image of nanoparticles loaded with imiquimod is shown in FIG. 3. Cryo transmission electron microscopy (cryo TEM) demonstrates that the majority of these nanoparticles are spherical with a unilamellar outer shell. The cryo TEM images also provide information related to the state of the encapsulated drug and the internal environment of the nanoparticles. The nanoparticles have interiors that are more dense than the surrounding buffer, consistent with the loading of the liposomes with imiquimod, HP-β-CD and PEG 4000 polymer in the interior of the liposome.

### Example 5: Non-Clinical Toxicity and Adjuvant Efficacy Studies

This example references studies which indicate that imiquimod as utilised in the presently disclosed nanoparticles is well tolerated in animals at doses greater than the clinical doses disclosed herein; and studies which demonstrate adjuvant activity of imiquimod at doses similar to the amounts of imiquimod delivered by way of the presently disclosed nanoparticles.

Various studies have shown that topical imiquimod is capable of enhancing the immune response induced by a vaccine. Pre-treatment with topical imiquimod also significantly improves the immunogenicity of influenza vaccination in both young and elderly individuals (Hung et al, Lancet Infect. Dis. 2016 February; 16 (2): 209-18). Similar results were reported by Adams et al, J. Clin. Oncol. 25 (18) suppl 8545, which evaluated the safety and adjuvant activity of imiquimod when administered with a NY-ESO-1 protein vaccine. The topical administration of imiquimod in these studies makes impossible an accurate assessment of the imiquimod dose delivered to the target immune cells.

In vitro treatment of macrophages with micelle encapsulated imiquimod produced significant activation of the NF-κB and MAPK pathways at concentrations of 0.2 μg/ml (Jiménez-Sánchez, et al 2014), indicating that local imiquimod concentrations of hundreds of ng/ml will activate the immune system. In vivo studies by Zhang et al (Clinical and Vaccine Immunology 2014. 21:4 pp570-579) found that 50 μg imiquimod injected intraperitoneally into mice, in combination with influenza vaccine significantly expedited and augmented the humoral immune responses against the virus and conferred significant protection mice against early lethal viral challenges.

The FDA Pharmacology/Toxicology review for Zyclara (imiquimod) cream 3.75% (Application Number 201153) describes a number of animal studies of the toxicology, pharmacokinetics, and metabolism of imiquimod in different species, and the findings from these studies include:
The subcutaneous lethal dose of a single subcutaneous dose in rats was 20 mg/kg.
Intravenous doses of 0.5 to 5.0 mg/kg imiquimod produced cardiac stimulation, central nervous system stimulation and some evidence of autonomic nervous system inhibition in dogs.
Intravenous doses of 0.5, 1 and 2 mg/kg/day imiquimod were administered to pregnant female rabbits during the period of organogenesis (gestational Days 6-18). No treatment related effects on embryofetal toxicity or teratogenicity were noted at 2 mg/kg/day.

The EMA's toxicology review of Aldara (imiquimod) 5% cream (https://www.ema.europa.eu/en/documents/scientific-discussion/aldara-epar-scientific-discussion_en.pdf) describes a number of animal studies of the toxicology, pharmacokinetics, and metabolism of imiquimod. It was reported that overall, “the toxicology program indicates a high degree of safety with no target organ toxicity other than that attributed to exaggerated pharmacological activity. Imiquimod did not affect fertility and it was neither teratogenic nor genotoxic. In carcinogenicity study in mice there was no increase in the incidence of tumors or non-neoplastic lesions as the result of dermal exposure to imiquimod”. Specific findings from these studies include:
Single dose toxicity of imiquimod studied in mice, rats, and monkeys indicated a high degree of safety. Adverse effects were limited to the central nervous system resulting in a number of clinical signs, usually convulsions, prior to death.
In two dermal toxicity studies in rabbits with doses of 2000 and 5000 mg/kg under occlusion there were no deaths and no signs of toxicity other than mild transient erythema at the application site.
Repeated dose toxicity of imiquimod after oral administration in rats and monkeys up to 6 months showed the only adverse effects apart from slight effects on body weight and food consumption were considered to be the result of exaggerated pharmacological activity, i.e., hyperplasia of B and T cell lymphoid tissue, increased number of plasma cells, enlargement of spleen and lymph nodes, Kupffer cell hyperplasia, mononuclear/macrophage cell accumulation or proliferation. There were no other target organs and a no observed adverse effect level (NOAEL) of 3 mg/kg was established in both species.

The maximum imiquimod dose of the nanoparticles disclosed herein are thousands-fold below the current dose levels of approved drugs or the MRHD based on numerous animal studies or a well tolerated 30 mg subcutaneous dose in healthy human volunteers (Soria, Myhre, et al., 2000). See also Table 3, which compares the quantity of imiquimod in a proposed 100 μl dose of the disclosed nanoparticles (NP) with the approved safe dose of Aldara® (imiquimod).

TABLE 3

| Imiquimod Dose | Imiquimod Safety Factor | Imiquimod Safety Factor for |
|---|---|---|
| (μg) in 100 μL | for 25 mg Aldara | 30 mg Human Subcutaneous Dose |
| Volume NP dose | MRHD mg (Single Dose) | (Soria, Myhre, et al., 2000) |
| 1 μg | 25,000-fold | 30,000-fold |

MRHD = maximum recommended human dose

Lipid nanoparticles having a hydrogel core, as utilised in embodiments of the present disclosure, have been shown to be preferentially taken up by antigen presenting cells (APCs), which may provide an advantage in immunological modulation. Look et al, Biomaterials 35 (2014) 1089-1095 showed that lipid nanoparticles having a hydrogel core were subject to more efficient uptake by dendritic cells in comparison with PLGA nanoparticles, with an >100× fold increase in uptake demonstrated by flow cytometry analysis and confocal imaging.

### Example 6: In Vitro Release Assays

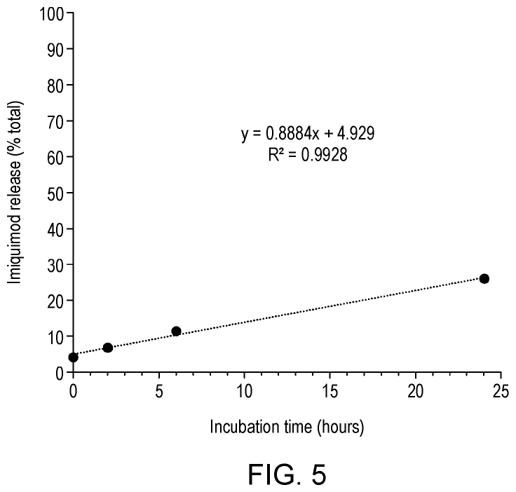

An in vitro release assay (IVRA) has been developed for imiquimod. Results of the IVRA are illustrated in FIG. 5. Here, nanoparticles produced according to Examples 1-3 were diluted with an equal volume of PBS buffer and incubated with gentle shaking at 37° C. Samples were removed at 0, 4, 6 and 24 hours and immediately processed using a 300 kd filter to obtain the released free (non-encapsulated) imiquimod in the filtrate. The concentration of imiquimod in the filtrate was determined using a reverse phase HPLC assay.

The results show that imiquimod is released from the nanolipogels at a linear rate over time, with approximately 25% of the drug released after 24 hours, and with complete release expected over a period of several days. This in vitro result demonstrates the ability of the nanolipogels to provide surprisingly sustained release and delivery of effective quantities of imiquimod over an extended time period of several days, which compares very favourably to prior art imiquimod formulations where the imiquimod was found to be released much more rapidly. Studies have shown that the immunostimulatory effects of TLR7/8 agonists such as imiquimod are enhanced by sustained delivery (Auderset et al Front Immunol. 2020 Nov. 11; 11: 580974). The delayed release achieved by the presently disclosed formulation therefore enhances the adjuvant effect of the formulation when administered as part of a vaccine.

### Example 7: Use in a Community Vaccination Setting

A dose of the vaccine composition of Example 3 is administered by parenteral intramuscular injection into the upper arm of a patient at risk of contracting COVID-19 disease. The adjuvant ingredients serve to enhance the protective immune response induced by the vaccine.

1. A vaccine adjuvant comprising a plurality of nanoparticles that comprise an outer lipid shell and an inner aqueous core encapsulated within the outer lipid shell;
wherein the inner aqueous core comprises an imidazoquinoline and a host molecule that is capable of reversibly forming a complex with the imidazoquinoline;
wherein the imidazoquinoline is an active TLR7/8 ligand having the molecular structure 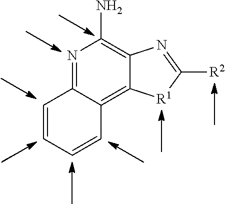
where R¹ is N and R² is H or C, and where the imidazoquinoline is unsubstituted or is substituted at one or more of the indicated addition points with one or more substituents, which substituents are independently selected from branched, linear or cyclic alkyl, alkenyl, alcohol, alkylamine, alkoxy or alkoxyalkyl groups, in particular, C₁₋₁₀ alkyl, alkenyl, alcohol, alkylamine, alkoxy or alkoxyalkyl groups, or hydroxyl groups, or amine groups, or N-(C₁₋₁₀ alkyl)methanesulphonamide groups;
and wherein the host molecule is a cyclodextrin.

2. The vaccine adjuvant of claim 1, wherein the imidazoquinoline is imiquimod (R-837), resiquimod, gardiquimod, S28690, 852-A, 854A, CL075 or CL097.

3. The vaccine adjuvant of claim 1, which comprises the imidazoquinoline complexed with the host molecule.

4. The vaccine adjuvant of claim 1, wherein the inner aqueous core of the nanoparticle comprises a hydrogel, which hydrogel is optionally selected from poly(lactic acid), poly(glycolic acid), poly(lactic acid-co-glycolic acids), polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); poly(glycolide-co-caprolactones); polycarbonates; polyamides, polypeptides, and poly(amino acids); polyesteramides; other biocompatible polyesters; poly(dioxanones); poly(alkylene alkylates); hydrophilic polyethers; polyurethanes; polyetheresters; polyacetals; polycyanoacrylates; polysiloxanes; poly(oxyethylene)/poly (oxypropylene) copolymers; polyketals; polyphosphates; polyhydroxyvalerates; polyalkylene oxalates; polyalkylene succinates; poly(maleic acids), polyvinyl alcohols, polyvinylpyrrolidone; poly(alkylene oxides); celluloses, polyacrylic acids, albumin, collagen, gelatin, prolamines, polysaccharides, derivatives, copolymers, and blends thereof.

5. The vaccine adjuvant of claim 1, wherein the host molecule is a cyclodextrin selected from α-cyclodextrin; β-cyclodextrin; γ-cyclodextrin; methyl α-cyclodextrin; methyl β-cyclodextrin; methyl γ-cyclodextrin; ethyl β-cyclodextrin; butyl α-cyclodextrin; butyl β-cyclodextrin; butyl γ-cyclodextrin; pentyl γ-cyclodextrin; hydroxyethyl β-cyclodextrin; hydroxyethyl γ-cyclodextrin; 2-hydroxypropyl α-cyclodextrin; 2-hydroxypropyl β-cyclodextrin; 2-hydroxypropyl γ-cyclodextrin; 2-hydroxybutyl β-cyclodextrin; acetyl α-cyclodextrin; acetyl β-cyclodextrin; acetyl γ-cyclodextrin; propionyl β-cyclodextrin; butyryl β-cyclodextrin; succinyl α-cyclodextrin; succinyl β-cyclodextrin; succinyl γ-cyclodextrin; benzoyl β-cyclodextrin; palmityl β-cyclodextrin; toluenesulfonyl β-cyclodextrin; acetyl methyl β-cyclodextrin; acetyl butyl β-cyclodextrin; glucosyl α-cyclodextrin; glucosyl β-cyclodextrin; glucosyl γ-cyclodextrin; maltosyl α-cyclodextrin; maltosyl β-cyclodextrin; maltosyl γ-cyclodextrin; α-cyclodextrin carboxymethylether; β-cyclodextrin carboxymethylether; γ-cyclodextrin carboxymethylether; carboxymethylethyl β-cyclodextrin; phosphate ester α-cyclodextrin; phosphate ester β-cyclodextrin; phosphate ester γ-cyclodextrin; 3-trimethylammonium-2-hydroxypropyl β-cyclodextrin; sulfobutyl ether β-cyclodextrin; carboxymethyl α-cyclodextrin; carboxymethyl β-cyclodextrin; carboxymethyl γ-cyclodextrin, and combinations thereof; advantageously, wherein the cyclodextrin is or includes 2-hydroxypropyl-β-cyclodextrin.

6. (canceled)

7. The vaccine adjuvant of claim 1, comprising an aqueous solution, aqueous dispersion or aqueous suspension of nanoparticles which is buffered to a pH of at least about 6.5, preferably at least pH 7; suitably between about pH 6.5-9 or between about pH 6.5-8.5 or between about pH 6.5-8 or between about pH 7-9 or between about pH 7-8.5 or between about pH 7-8 or between about pH 7.5-9.

8-10. (canceled)

11. A vaccine composition comprising:
(a) an antigen that is capable of inducing an immune response and/or a polynucleotide that encodes an antigen capable of inducing an immune response; and
(b) the vaccine adjuvant of claim 1.

12. (canceled)

13. The vaccine composition of claim 11, wherein some or all of the antigen and/or polynucleotide is releasably attached to, associated with and/or encapsulated within the outer lipid shell of the nanoparticles.

14. The vaccine composition of claim 11, wherein the polynucleotide is a DNA molecule or an RNA molecule, such as an mRNA molecule; and/or wherein the antigen is a viral, bacterial, fungal or disease- or cancer-associated antigen; and/or wherein the polynucleotide encodes a viral, bacterial, fungal or disease- or cancer-associated antigen.

15-16. (canceled)

17. The vaccine composition of claim 11, wherein the antigen is a coronavirus or coronavirus-associated antigen, such as SARS-CoV, MERS-CoV or SARS-CoV-2 antigen; or an influenza or influenza-associated antigen, such as an influenza A, influenza B, influenza C or influenza D antigen; or a Herpes simplex (HSV-1 or HSV-2) or HSV-associated antigen; or a cytomegalovirus (CMV) or CMV-associated antigen; or a Lyme's Disease (Borrelia) or Lyme's Disease-associated antigen; or a Respiratory Syncytial Virus (RSV) or RSV-associated antigen; or an Epstein-Barr Virus (EBV) or EBV-associated antigen; or a Zika virus or Zika virus-associated antigen; or a meningitis or meningitis-associated antigen; or a measles or measles-associated antigen; or a mumps or mumps-associated antigen; or a rubella or rubella-associated antigen; or a varicella (chickenpox) or chickenpox-associated antigen; or a Herpes zoster (shingles) or shingles-associated antigen; or a diphtheria or diphetheria-associated antigen; or a tetanus or tetanus-associated antigen; or a poliomyelitis or poliomyelitis-associated antigen; or a dengue virus or dengue virus-associated antigen; or a Haemophilus influenzae (Hib) or Hib-associated antigen; or a rotavirus or rotavirus-associated antigen; or a Streptococcus pneumoniae (Streptococcus) or Streptococcus-associated antigen; or a human papillomavirus (HPV) or HPV-associated antigen; or a pertussis or pertussis-associated antigen; or a hepatitis or hepatitis-associated antigen; or a tuberculosis or tuberculosis-associated antigen; or a human immunodeficiency virus (HIV) or HIV-associated antigen; or an adenovirus or adenovirus-associated antigen; or an anthrax or anthrax-associated antigen; or a cholera or cholera-associated antigen; or a Japanese Encephalitis (JE) or JE-associated antigen; or a rabies or rabies-associated antigen; or a smallpox or smallpox-associated antigen; or a Typhoid Fever (typhoid) or typhoid-associated antigen; or a yellow fever or yellow fever-associated antigen; or an Ebola or Ebola-associated antigen; or a cancer or cancer-associated antigen; and/or wherein the polynucleotide encodes an antigen which is a coronavirus or coronavirus-associated antigen, such a SARS-CoV, MERS-CoV or SARS-CoV-2 antigen; or an influenza or influenza-associated antigen, such as an influenza A, influenza B, influenza C or influenza D antigen; or a Herpes simplex (HSV-1 or HSV-2) or HSV-associated antigen; or a cytomegalovirus (CMV) or CMV-associated antigen; or a Lyme's Disease (Borrelia) or Lyme's Disease-associated antigen; or a Respiratory Syncytial Virus (RSV) or RSV-associated antigen; or an Epstein-Barr Virus (EBV) or EBV-associated antigen; or a Zika virus or Zika virus-associated antigen; or a meningitis or meningitis-associated antigen; or a measles or measles-associated antigen; or a mumps or mumps-associated antigen; or a rubella or rubella-associated antigen; or a varicella (chickenpox) or chickenpox-associated antigen; or a Herpes zoster (shingles) or shingles-associated antigen; or a diphtheria or diphetheria-associated antigen; or a tetanus or tetanus-associated antigen; or a poliomyelitis or poliomyelitis-associated antigen; or a dengue virus or dengue virus-associated antigen; or a Haemophilus influenzae (Hib) or Hib-associated antigen; or a rotavirus or rotavirus-associated antigen; or a Streptococcus pneumoniae (Streptococcus) or Streptococcus-associated antigen; or a human papillomavirus (HPV) or HPV-associated antigen; or a pertussis or pertussis-associated antigen; or a hepatitis or hepatitis-associated antigen; or a tuberculosis or tuberculosis-associated antigen; or a human immunodeficiency virus (HIV) or HIV-associated antigen; or an adenovirus or adenovirus-associated antigen; or an anthrax or anthrax-associated antigen; or a cholera or cholera-associated antigen; or a Japanese Encephalitis (JE) or JE-associated antigen; or a rabies or rabies-associated antigen; or a smallpox or smallpox-associated antigen; or a Typhoid Fever (typhoid) or typhoid-associated antigen; or a yellow fever or yellow fever-associated antigen; or an Ebola or Ebola-associated antigen; or a cancer or cancer-associated antigen.

18. (canceled)

19. A method for inducing an immune response in a subject, comprising administering to the subject the vaccine composition of claim 11.

20. (canceled)

21. A method for immunising a subject against a viral, bacterial or fungal infection, colonisation or disease, or against a proliferative disorder such as cancer comprising administering to the subject the vaccine composition of claim 11.

22. A method for manufacturing the vaccine adjuvant of claim 1, comprising the sequential steps of:
(a) solubilising the imidazoquinoline with the host molecule in aqueous solution at a pH buffered to about pH 6 or below; preferably to about pH 4-6, or to about pH 4.5-6, or to about pH 4-5.5, or to about pH 5-6;
(b) combining the resulting aqueous solution with lipids to form lipid shell nanoparticles encapsulating the imidazoquinoline; and
(c) increasing the buffered pH of the formulation to about pH 6.5 or above, or to pH 7 or above, or to about pH 6.5-9 or to about pH 6.5-8.5, or to about pH 6.5-8; or to about pH 7-9 or to about pH 7-8.5 or to about pH 7-8 or to about pH 7.5-9.

23. The method of claim 22, wherein step (b) comprises mixing the solution of (a) with lipids, which are optionally solubilised in alcoholic solution, to form a solution or suspension of multilamellar structures, and processing these structures to form lipid shell nanoparticles encapsulating the imidazoquinoline.

24-26. (canceled)

27. The method of claim 22, wherein step (a) comprises solubilising the imidazoquinoline in aqueous solution in the presence of a hydroxyacid, such as citric acid, tartaric acid, lactic acid, glycolic acid or malic acid, optionally in the presence of a host molecule such as a cyclodextrin.

28. The method of claim 22, wherein step (a) involves combining the imidazoquinoline with a host molecule such as cyclodextrin in solution at a pH between about 4-5.5; preferably at a pH of about 5.

29. The method of claim 22, further comprising a step of adding a hydrogel polymer, such as a PEG polymer, during or after step (a).

30. The method of claim 22, further comprising reducing unencapsulated the imidazoquinoline from the nanoparticle formulation of (b), after step (b) and before step (c), optionally by ultracentrifugation or by diafiltration with membranes which are sized to retain the nanoparticles but permit the passage of free the imidazoquinoline.

31. (canceled)

32. A method for producing a vaccine composition comprising combining an antigen that is capable of inducing an immune response and/or a polynucleotide that encodes an antigen capable of inducing an immune response with the vaccine adjuvant of claim 1.

33-35. (canceled)
